(19) United States
(12) Patent Application Publication
(10) Pub. No.: US 2018/0200059 A1
(43) Pub. Date: Jul. 19, 2018

(54) SADDLE ANNULOPLASTY WITH PROGRESSIVE 2D SHAPE

(71) Applicant: The Trustees of The University of Pennsylvania, Philadelphia, PA (US)

(72) Inventors: Joseph H. GORMAN, III, Lower Gwynedd, PA (US); Robert C. GORMAN, Lower Gwynedd, PA (US)

(21) Appl. No.: 15/743,780

(22) PCT Filed: Jul. 11, 2016
(86) PCT No.: PCT/US16/41733

§ 371 Date: Jan. 11, 2018

Related U.S. Application Data
(60) Provisional application No. 62/192,862, filed on Jul. 15, 2015.

(51) Int. Cl. A61F 2/24

(52) U.S. Cl. CPC A61F 2/2448; A61F 2/2415

(57) ABSTRACT

Disclosed are annuloplasty rings having defined relationships between the intercommisural width and ratio of septo-lateral width to intercommisural width. The saddle-shaped annuloplasty rings may have a ratio of SLW to ICW of x±(0.07×x) when the ring has an intercommisural width of y, as determined according to the formula x=(y−124)/−100.

### CROSS REFERENCE TO RELATED APPLICATIONS

This application claims the benefit of priority to U.S. Provisional App. No. 62/192,862, filed Jul. 15, 2015, the entire contents of which are incorporated herein by reference.

### GOVERNMENT RIGHTS

This invention was made with government support under HL R01-073021 (J. H. Gorman III, M.D.), awarded by National Institutes of Health. The government has certain rights in the invention.

### TECHNICAL FIELD

The present disclosure relates to annuloplasty ring design.

### BACKGROUND

The mitral valve is a complex structure whose competence relies on the precise interaction of annulus, leaflets, chordae, papillary muscles, and left ventricle (LV). Pathologic changes in any of these structures can lead to valvular insufficiency. Myxomatous leaflet/chordal degeneration, and dilated ischemic cardiomyopathy secondary to chronic post infarction ventricular remodeling are among most common mechanisms producing mitral regurgitation (MR). These two disease processes account for about 78% of all cases of MR treated surgically.

Ring annuloplasty was recognized as an essential component of mitral valve repair at least as early as 1969. The goal of an annuloplasty ring (sometimes referred to simply as “ring”) is to remodel the annulus back to a more normal geometry, decreasing tension on suture lines, increasing leaflet coaptation and preventing progressive annular dilatation. Surgeons with experience in mitral valve repair agree that restoration of normal annular shape with a ring is an essential component to most if not all mitral valve repairs. In fact it has been shown that repairs without a ring are less durable.

It is now known that ring geometry is critical to addressing the problems caused by leaflet stresses. See Salgo I S, et al., “Effect of Annular Shape on Leaflet Curvature in Reducing Mitral Leaflet Stress,” Circulation, Vol. 106, pp. 711-17 (2002). Salgo, et al., reported that, inter alia, a saddle-shaped ring and other criteria can be used in the design and fabrication of cardiac valvular support prostheses. Subsequent annuloplasty ring designs by Medtronic (Profile 3D Annuloplasty Ring, 2008) and Carpentier (Physio II Annuloplasty Ring, 2009) adopted the saddle-shaped annuloplasty form.

There remains a need for other criteria for the design of annuloplasty rings that improve the ability of valve prostheses to return hearts to normal functioning and to maintain such a state over time.

### SUMMARY

Provided herein are methods for making a saddle-shaped annuloplasty ring having a septo-lateral width (SLW) and an intercommisural width (ICW) comprising selecting the ratio of SLW to ICW so that said ratio is lower than about 1.0 when said ring has an ICW of about 24 or more, and said ratio is higher than about 0.8 when said ring has an ICW of about 44 or less.

Also disclosed are saddle-shaped annuloplasty rings having a septo-lateral width (SLW), an intercommisural width (ICW), and a ratio of SLW to ICW of x±(0.07×x) when said rings have an ICW of y, as determined according to the formula x=(y−124)/−100.

The present disclosure also relates to methods of making a saddle-shaped annuloplasty ring having a septo-lateral width (SLW), and an intercommisural width (ICW), comprising forming a ring having a ratio of SLW to ICW of x′ when said ring has a ICW of y:

| x′ | y |
|---|---|
| 1.0 ± 0.07 | 24 |
| 0.97 ± 0.07 | 27 |
| 0.94 ± 0.07 | 30 |
| 0.91 ± 0.07 | 33 |
| 0.88 ± 0.07 | 36 |
| 0.85 ± 0.07 | 39 |
| 0.82 ± 0.07 | 42 |
| 0.79 ± 0.07 | 45 |

Also disclosed are saddle-shaped annuloplasty rings having a septo-lateral width (SLW), and an intercommisural width (ICW), and a ratio of SLW to ICW of x′ when said ring has a ICW of y:

| x | y |
|---|---|
| 1.0 ± 0.07 | 24 |
| 0.97 ± 0.07 | 27 |
| 0.94 ± 0.07 | 30 |
| 0.91 ± 0.07 | 33 |
| 0.88 ± 0.07 | 36 |
| 0.85 ± 0.07 | 39 |
| 0.82 ± 0.07 | 42 |
| 0.79 ± 0.07 | 45 |

The present disclosure also provides kits comprising a plurality of saddle-shaped annuloplasty rings, each of said rings having a septo-lateral width (SLW) and an intercommisural width (ICW), wherein at least two of said rings vary according to ICW, and wherein each of said rings have a ratio of SLW to ICW of x±(0.07×x) and an ICW of y, as determined according to the formula x=(y−124)/−100.

Also disclosed are methods for assembling a kit comprising providing a plurality of saddle-shaped annuloplasty rings, each of said rings having a septo-lateral width (SLW) and an intercommisural width (ICW), at least two of said rings vary according to ICW, and wherein each of said rings have a ratio of SLW to ICW of x±(0.07×x) and an ICW of y, as determined according to the formula x=(y−124)/−100.

### BRIEF DESCRIPTION OF THE DRAWINGS

FIG. 1 provides a diagram of a landmarked contour for the mitral valve annulus.

FIG. 2 illustrates how, pursuant to the present invention, an increase in ICW and commensurate decrease in SLW/ICW requires the use of a more elliptical (e.g., a more D-shaped) annuloplasty ring, whereas a decrease in ICW and commensurate increase in SLW/ICW requires a more circular annuloplasty ring.

FIG. 3 shows a general trendline of one possible relationship between ICW (y-axis) and SLW/ICW (x-axis) according to the present invention.

### DETAILED DESCRIPTION OF ILLUSTRATIVE EMBODIMENTS

The present inventions may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a particle” is a reference to one or more of such particles and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

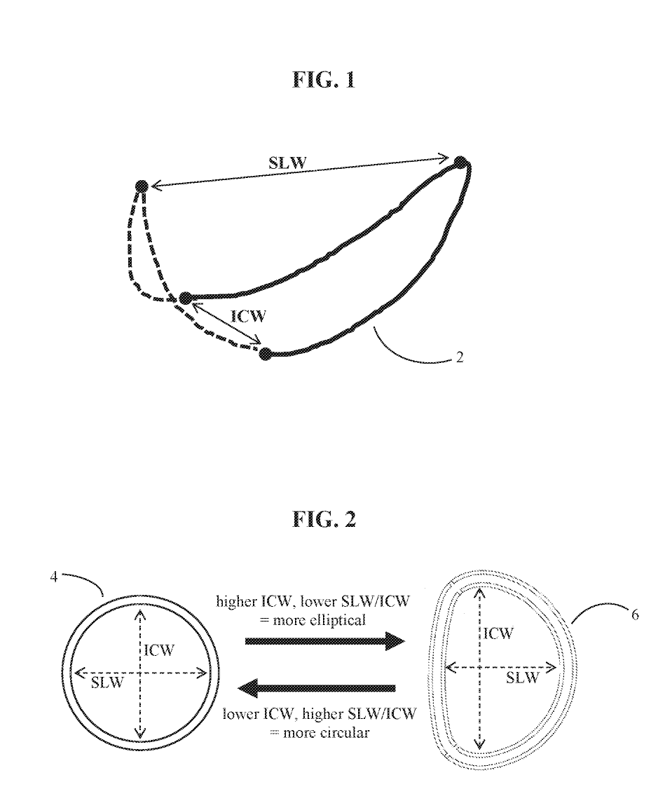

The present disclosure relates to saddle-shaped annuloplasty rings having a ratio of septo-lateral width (SLW) to intercommisural width (ICW) that is tailored to the size of a specific subject's mitral valve annulus. FIG. 1 provides a diagram of the landmarked contour 2 for the mitral valve annulus. The anterior annulus is shown as dashed lines and posterior annulus is shown as solid lines. In the diagram, the septo-lateral width (SLW) and the intercommisural width (ICW) are shown using respective double-headed arrows.

Much like other anatomical features, the mitral valve annulus can vary in shape and size from person to person. Since the publication of Salgo I S, et al. (cited in full above), whose authors were the first to recognize the significance of the geometry of the mitral valve annulus to leaflet function and integrity, it has been known that both the three dimensional shape and the two dimensional sizes of annuloplasty rings should also vary so that they correspond to the specific needs of a particular patient. Although previous efforts have been made to vary the geometrical characteristics of annuloplasty rings in terms of, for example, the annular height to commissural width ratio (thereby permitting variation with respect to the curvature of the hyperbolic paraboloid that produces the saddle shape of the ring) and the SLW/ICW ratio relative to ICW, with respect to the characteristic of two dimensional shape, it has classically been believed that as the intercommisural width of the mitral valve annulus increased, the shape of the annulus should trend towards circular, and away from the elliptical or D-shape. Edwards Lifesciences currently produces mitral valve annuloplasty rings referred to as “Carpentier-Edwards Physio II Annuloplasty Rings” that vary by shape, and wherein the rings with smaller ICW have a more elliptical-shaped two dimensional profile, and the rings with greater ICW have a more circular (but still elliptical) two dimensional profile. For example, the Physio II rings having an intercommisural width of 40 (representing rings for use in patients with larger than average annuli) have a ratio of septo-lateral width to intercommisural width of 0.80, thereby representing a ring that is moderately elliptical. The Physio II rings having an intercommisural width of 24 (representing rings for use in patients with smaller than average annuli) have a ratio of septo-lateral width to intercommisural width of 0.6875, representing a ring that is more strongly elliptical. Thus, the trend among the Physio II rings is that smaller rings are more elliptical, and larger rings are less elliptical.

The present inventors have surprisingly discovered that the opposite of the traditional belief is true, i.e., that smaller mitral valve annuli require more circular annuloplasty rings, and that progressively more elliptical rings (for example, trending towards D-shaped) should be provided for larger annuli. Stated differently, as intercommisural width of the native annulus increases, and the ratio of septo-lateral width to intercommisural width (SLW/ICW) decreases, a more elliptical annuloplasty ring is required. Likewise, as the size of the annulus, as expressed in terms of the intercommisural width, decreases, the ratio of septo-lateral width to intercommisural width (SLW/ICW) increases, a more circular annuloplasty ring is required. The smallest native mitral annuli, for example, can require an essentially perfectly circular annuloplasty ring.

FIG. 2 illustrates how, pursuant to the present invention, an increase in ICW and commensurate decrease in SLW/ICW requires the use of a more elliptical annuloplasty ring 6, whereas a decrease in ICW and commensurate increase in SLW/ICW requires a more circular annuloplasty ring 4. For simplicity, only the two-dimensional shape is shown—in all embodiments of the present invention, the annuloplasty ring should adopt a saddle shape in its three dimensional aspect.

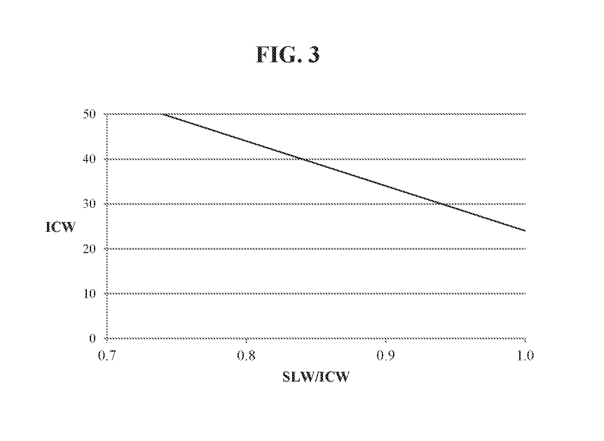

FIG. 3 shows a general trendline of one possible relationship between ICW (y-axis) and SLW/ICW (x-axis) according to the present invention. Pursuant to the depicted trendline, the ratio of septo-lateral width to intercommisural width (SLW/ICW) can be determined according to the formula x=(y−124)/−100, wherein x is SLW/ICW and y is ICW. The x values that are respectively obtained from a particular y value in accordance with this formula can vary by about 4%, about 5%, about 6%, or about 7%. For example, for an ICW of 44, the corresponding SLW/ICW ratio may be 0.8±0.04, when a variation of about 5% applies. As used herein, the phrase x±(0.07×x) means that x may be any number within the expressed range of variation, in this case any number that falls within the range of x±(0.07×x). Thus, the phrase x±(0.07×x) embraces values corresponding to x±(0.06×x), x±(0.05×x), x±(0.04×x), and the like.

In accordance with the preceding, provided herein are methods for making a saddle-shaped annuloplasty ring having a septo-lateral width (SLW) and an intercommisural width (ICW) comprising selecting the ratio of SLW to ICW so that said ratio is lower than about 1.0 when said ring has an ICW of about 24 or more, and said ratio is higher than about 0.8 when said ring has an ICW of about 44 or less. In accordance with such methods, the ratio of SLW to ICW may be x±(0.07×x) when the ring has an ICW of y, as determined according to the formula x=(y−124)/−100. In some embodiments, the ratio of SLW to ICW is x±(0.06×x), x±(0.05×x), or x±(0.04×x). In certain instances the present methods comprise selecting the ratio of SLW to ICW so that said ratio is lower than about 1.0 but higher than about 0.75 when said ring has an ICW of about 24 or more, and said ratio is higher than about 0.8 but lower than about 1.0 when said ring has an ICW of about 44 or less.

The annuloplasty rings according to each of the presently disclosed embodiments may be manufactured using materials and equipment with which those of ordinary skill in the art are familiar. The three-dimensional, i.e., saddle-shaped, characteristics of the presently disclosed rings may be provided in accordance with known parameters, for example, in accordance with the parameters described in U.S. Pub. No. 2008/0154359, the entire contents of which are incorporated herein by reference.

Also disclosed are saddle-shaped annuloplasty rings having a septo-lateral width (SLW), an intercommisural width (ICW), and a ratio of SLW to ICW of x±(0.07×x) when the ring has an ICW of y, as determined according to the formula x=(y−124)/−100. In some embodiments of the present rings, the ratio of SLW to ICW is x±(0.06×x), x±(0.05×x), or x±(0.04×x).

The present disclosure also provides methods of making a saddle-shaped annuloplasty ring having a septo-lateral width (SLW) and an intercommisural width (ICW), comprising forming a ring having a ratio of SLW to ICW of x′ when said ring has a ICW of y, in accordance with Table 1:

TABLE 1

| x′ | y |
|---|---|
| 1.0 ± 0.07 | 24 |
| 0.97 ± 0.07 | 27 |
| 0.94 ± 0.07 | 30 |
| 0.91 ± 0.07 | 33 |
| 0.88 ± 0.07 | 36 |
| 0.85 ± 0.07 | 39 |
| 0.82 ± 0.07 | 42 |
| 0.79 ± 0.07 | 45 |

Also provided herein are saddle-shaped annuloplasty rings having a septo-lateral width (SLW), an intercommisural width (ICW), and a ratio of SLW to ICW of x′ when said ring has a ICW of y, in accordance with the values shown in Table 1.

The present disclosure also relates to kits comprising a plurality of saddle-shaped annuloplasty rings, each of said rings having a septo-lateral width (SLW) and an intercommisural width (ICW), wherein at least two of said rings vary according to their respective ICW values, and wherein each of said rings have a ratio of SLW to ICW of x±(0.07×x) and an ICW of y, as determined according to the formula x=(y−124)/−100. In some embodiments, at least three, at least four, at least five, or at least six of the rings in the kit vary according to their respective ICW values.

Also provided are methods for assembling a kit comprising providing a plurality of saddle-shaped annuloplasty rings, each of the rings having a septo-lateral width (SLW) and an intercommisural width (ICW), at least two of the rings vary according to their respective ICW values, and wherein each of the rings have a ratio of SLW to ICW of x±(0.07×x) and an ICW of y, as determined according to the formula x=(y−124)/−100. In some embodiments, at least three, at least four, at least five, or at least six of the rings in the kit vary according to their respective ICW values.

The following example is set forth so as to provide those of ordinary skill in the art with a complete disclosure and description of how the annuloplasty rings claimed herein may be made, and are intended to be purely exemplary of the invention and are not intended to limit the scope of what the inventors regard as their invention.

### Example 1—Kit

A kit comprising a plurality of annuloplasty rings is assembled as follows. A first saddle-shaped annuloplasty ring having an ICW of 24 and a SLW to ICW ratio of 1.0 is placed within a sterile packaging material, which is sealed so that it is substantially impervious to biological contamination. A second saddle-shaped annuloplasty ring having an ICW of 31 and a SLW to ICW ratio of 0.96 is also placed within a sterile packaging material, which is sealed so that it is substantially impervious to biological contamination. A third saddle-shaped annuloplasty ring having an ICW of 35 and a SLW to ICW ratio of 0.89 is also placed within sterile packaging. A fourth saddle-shaped annuloplasty ring having an ICW of 39 and a SLW to ICW ratio of 0.85 is also packaged as described above. A fifth saddle-shaped annuloplasty ring having an ICW of 42 and a SLW to ICW ratio of 0.83 is likewise sterile-packaged. A sixth saddle-shaped annuloplasty ring having an ICW of 44 and a SLW to ICW ratio of 0.80 is sterile-packaged in the same manner. Each of the six packaged annuloplasty rings are placed within an additional packaging material, along with instructions for the use of the rings. The resulting kit is suitable for shipping to a hospital or medical practitioner.

What is claimed:

1. A method for making a saddle-shaped annuloplasty ring having a septo-lateral width (SLW) and an intercommisural width (ICW) comprising selecting the ratio of SLW to ICW so that said ratio is lower than about 1.0 when said ring has an ICW of about 24 or more, and said ratio is higher than about 0.8 when said ring has an ICW of about 44 or less.

2. The method according to claim 1 wherein the ratio of SLW to ICW is x±(0.07×x) when said ring has an ICW of y, as determined according to the formula x=(y−124)/−100.

3. The method according to claim 2 wherein the ratio of SLW to ICW is x±(0.06×x).

4. The method according to claim 2 wherein the ratio of SLW to ICW is x±(0.05×x).

5. The method according to claim 1 comprising selecting the ratio of SLW to ICW so that said ratio is lower than about 1.0 but higher than about 0.75 when said ring has an ICW of about 24 or more, and said ratio is higher than about 0.8 but lower than about 1.0 when said ring has an ICW of about 44 or less.

6. A saddle-shaped annuloplasty ring having a septo-lateral width (SLW), an intercommisural width (ICW), and a ratio of SLW to ICW of x±(0.07×x) when said ring has an ICW of y, as determined according to the formula x=(y−124)/−100.

7. The annuloplasty ring according to claim 6 wherein the ratio of SLW to ICW is x±(0.06×x).

8. The annuloplasty ring according to claim 6 wherein the ratio of SLW to ICW is x±(0.05×x).

9. A method of making a saddle-shaped annuloplasty ring having a septo-lateral width (SLW), an intercommisural width (ICW) comprising forming a ring having a ratio of SLW to ICW of x′ when said ring has a ICW of y:

| x′ | y |
|---|---|
| 1.0 ± 0.07 | 24 |
| 0.97 ± 0.07 | 27 |
| 0.94 ± 0.07 | 30 |
| 0.91 ± 0.07 | 33 |
| 0.88 ± 0.07 | 36 |
| 0.85 ± 0.07 | 39 |
| 0.82 ± 0.07 | 42 |
| 0.79 ± 0.07 | 45 |

10. A saddle-shaped annuloplasty ring having a septo-lateral width (SLW), an intercommisural width (ICW) comprising forming a ring having a ratio of SLW to ICW of x′ when said ring has a ICW of y:

| X | y |
|---|---|
| 1.0 ± 0.07 | 24 |
| 0.97 ± 0.07 | 27 |
| 0.94 ± 0.07 | 30 |
| 0.91 ± 0.07 | 33 |
| 0.88 ± 0.07 | 36 |
| 0.85 ± 0.07 | 39 |
| 0.82 ± 0.07 | 42 |
| 0.79 ± 0.07 | 45 |

11. A kit comprising a plurality of saddle-shaped annuloplasty rings, each of said rings having a septo-lateral width (SLW) and an intercommisural width (ICW), wherein at least two of said rings vary according to their respective ICW values, and wherein each of said rings have a ratio of SLW to ICW of x±(0.07×x) and an ICW of y, as determined according to the formula x=(y−124)/−100.

12. A method for assembling a kit comprising providing a plurality of saddle-shaped annuloplasty rings, each of said rings having a septo-lateral width (SLW) and an intercommisural width (ICW), at least two of said rings vary according to their respective ICW values, and wherein each of said rings have a ratio of SLW to ICW of x±(0.07×x) and an ICW of y, as determined according to the formula x=(y−124)/−100.
